(19) United States
(12) Patent Application Publication
(10) Pub. No.: US 2004/0204363 A1
(43) Pub. Date: Oct. 14, 2004

(54) Use of heat shock proteins to deliver moieties into cells

(75) Inventor: Richard A. Young, Weston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 10/843,659

(22) Filed: May 11, 2004

Related U.S. Application Data
(63) Continuation of application No. 09/756,543, filed on Jan. 8, 2001.
Application No. 09/756,543 is a continuation of application No. 09/025,178, filed on Feb. 18, 1998, now abandoned.
(60) Provisional application No. 60/038,059, filed on Feb. 18, 1997.
(60) Provisional application No. 60/066,288, filed on Nov. 25, 1997.

(51) Int. Cl. A61K 38/17

(52) U.S. Cl. 514/12; 514/8

(57) ABSTRACT

The present invention relates to a method of delivering a moiety of interest into a cell comprising contacting the cell with a complex comprising the moiety of interest covalently linked to a heat shock protein, under conditions appropriate for entry of the complex into the cell. The invention also relates to a method of delivering a moiety of interest into a cell capable of taking up a complex comprising the moiety of interest covalently linked to a heat shock protein comprising contacting the cell with a complex comprising the moiety of interest covalently linked to a heat shock protein, under conditions appropriate for entry of the complex into the cell. Also encompassed by the present invention is a method of delivering a moiety of interest into an antigen presenting cell comprising contacting the cell with a complex comprising the moiety of interest covalently linked to a heat shock protein, under conditions appropriate for entry of the complex into the cell.

### RELATED APPLICATION(S)

This application is a continuation of U.S. application Ser. No. 09/756,543, filed Jan. 8, 2001, which is a continuation of U.S. application Ser. No. 09/025,178, filed Feb. 18, 1998, which claims the benefit of U.S. Provisional Application No. 60/038,059, filed Feb. 18, 1997 and U.S. Provisional Application No. 60/066,288, filed Nov. 25, 1997. The entire teachings of the above applications are incorporated herein by reference.

### GOVERNMENT SUPPORT

The invention was supported, in whole or in part, by grants AI26463 and AI31869 from The National Institutes of Health. The Government has certain rights in the invention.

### BACKGROUND OF THE INVENTION

The cytotoxic T lymphocytes (CTL) that play an important role in protective cellular immunity, including the destruction of virus-infected cells, are predominantly CD8 T cells (Byrne, J. A. & Oldstone, M. B., J. Virol., 51:682-686 (1984); Nagler-Anderson, C. et al., J. Immunol., 141:3299-3305 (1988)). Antigen-specific activation of these cells depends upon their recognition of peptide-MHC complexes, which normally arise within antigen presenting cells by proteolytic cleavage of cytosolic proteins (Townsend, A. & Bodmer, H., Annu. Rev. Immuno., 7:601-624 (1989)). Translocated into the ER, the resulting peptides bind to nascent class I MHC molecules for transport to the cell surface (Heemels, M. T. & Ploegh, H., Annu. Rev. Biochem., 64:463-491 (1995)). However, many intact and/or functional molecules such as proteins cannot ordinarily penetrate into a cell's cytosol on their own.

### SUMMARY OF THE INVENTION

The present invention relates to a method of delivering a moiety of interest (e.g., protein, lipid) into a cell comprising contacting the cell with a complex comprising the moiety of interest covalently linked to a heat shock protein (hsp) (e.g., a mycobacterial hsp), under conditions appropriate for entry of the complex into the cell. The complex can comprise the moiety of interest conjugated to the hsp. Alternatively, the complex can comprise the moiety fused to the hsp. These two embodiments of complexes of the present invention are referred to, respectively, as hsp-moiety of interest conjugates and hsp-moiety of interest fusions.

In one embodiment, the present invention relates to a method of delivering a moiety of interest into a cell capable of taking up a complex comprising the moiety of interest covalently linked to a heat shock protein, comprising contacting the cell with the complex, under conditions appropriate for entry of the complex into the cell.

In another embodiment, the present invention relates to a method of delivering a moiety of interest into an antigen presenting cell comprising contacting the cell with a complex comprising the moiety of interest covalently linked to a heat shock protein, under conditions appropriate for entry of the complex into the cell.

The present invention also relates to a method of delivering a moiety of interest into a cell of an individual (e.g., human) comprising contacting the cell with a complex comprising the moiety of interest covalently linked to a heat shock protein, under conditions appropriate for entry of the complex into the cell.

In one embodiment, the present invention relates to a method of delivering a moiety of interest into a cell of an individual wherein the cell is capable of taking up a complex comprising the moiety of interest covalently linked to a heat shock protein, comprising contacting the cell with the complex, under conditions appropriate for entry of the complex into the cell.

In another embodiment, the invention relates to a method of delivering a moiety of interest into an antigen presenting cell of an individual comprising contacting the cell with a complex comprising the moiety of interest covalently linked to a heat shock protein, under conditions appropriate for entry of the complex into the cell.

### BRIEF DESCRIPTION OF THE DRAWINGS

FIG. 1A is a graph of effector cells to target cells (E:T) ratios versus % specific lysis showing generation of ovalbumin-specific CTL by immunization with ova-hsp70 fusion protein in saline.

FIG. 1B is a graph of log [SIINFEKL] versus % specific lysis showing a SIINFEKL peptide (SEQ ID NO: 1) titration, wherein T2-K^{b} cells were incubated with the indicated molar concentrations of SIINFEKL peptide (SEQ ID NO: 1) for 45 minutes for use as target cells in a CTL assay.

FIGS. 2A-2C are graphs of E:T ratios versus % specific lysis demonstrating that immunization with ova-hsp70 elicits ovalbumin reactive CD8⁺ T cells.

FIG. 3A is a bar graph showing IFN-γ secretion by splenocytes stimulated 72 hours in vitro with 5 μg/ml recombinant ova protein ▪, SIINFEKL peptide (SEQ ID NO: 1) (hatched box), RGYVYQGL peptide (SEQ ID NO: 2) (lightly shaded box), or tissue culture media alone □; all samples were examined in triplicate.

FIG. 3B is a graph of E:T ratios versus % specific lysis showing generation of ova-specific CTL by immunization with ova-hsp70 fusion protein in saline.

FIGS. 4A-4B are graphs of days versus tumor diameter, wherein, following the M05 (FIG. 4A) and B16 (FIG. 4B) tumor challenges, tumor growth was monitored in control mice Δ and in ova □ and ova-hsp70 ▪ immunized mice, and recorded as the average tumor diameter in millimeters.

FIG. 4C is a graph of days versus % survival wherein the survival of mice was recorded as the percentage of mice surviving following the tumor challenge; mice which appeared moribund were killed and scored as ‘not surviving’.

### DETAILED DESCRIPTION OF THE INVENTION

A description of preferred embodiments of the invention follows. The present invention relates to a method of delivering moieties or molecules (e.g., proteins, peptides, lipids) which are not generally able to enter cells or which enter cells only to a limited extent, into cells or into cells of an individual, and to complexes, including hsp-moiety of interest conjugates and hsp-moiety of interest fusions, such as protein complexes or fusion proteins, useful in the method. As a result of the present method, a functional molecule (e.g., a biologically active molecule) is delivered into cells. As described herein, Applicant has shown that covalently coupling a heat shock protein (hsp), such as a mycobacterial hsp, to a moiety which cannot enter mammalian cells on its own or which enters mammalian cells on its own only to a limited extent, results in delivery of the moiety into cells. As described herein, the ability of an hsp present in a complex comprising the hsp linked to a moiety of interest, to elicit MHC class I-restricted CTLs against the attached moiety indicates that the complex is able to enter cells, as an intact molecule, and enter the class I antigen presentation pathway of the cell. Thus, the methods of the present invention can be used to deliver a moiety which is not generally able to enter cells or which enters cells only to a limited extent, into cells (e.g., of an individual) which are able to take up the complexes (such as cells having an MHC class I antigen presentation pathway).

Moieties such as proteins, peptides, lipids, glycoproteins, small organic molecules and other molecules, particularly chemicals, and other molecules which are useful therapeutically or diagnostically, are delivered into mammalian cells by the present method. For example, a fusion protein comprising a hsp linked or coupled to a moiety to be delivered into cells is administered to/introduced into a mammal, such as a mouse, monkey or human, as a soluble protein using known techniques and routes of administration. Alternatively, an hsp-moiety of interest conjugate can be introduced into cells. The moiety to be delivered enters cells as a result of the ability of the hsp component to enter cells or chaperone entry of the moiety into cells.

As described herein, a complex comprising a moiety of interest and an hsp is delivered into cells. The hsp can be conjugated or joined to the moiety of interest to form a single unit. In one embodiment, the hsp is conjugated to the moiety of interest, such as by chemical means, to produce an hsp-moiety of interest conjugate. In another embodiment, the hsp is fused to the moiety of interest, such as by recombinant techniques (e.g., expression of the hsp and moiety of interest by recombinant DNA techniques). Conjugation can be achieved by chemical means known to those skilled in the art (e.g., through a covalent bond between the hsp and the moiety; reductive amination). If recombinant techniques are used to link the hsp and the moiety, the result is a recombinant fusion protein which includes the hsp and the moiety in a single molecule. This makes it possible to produce and purify a single recombinant molecule.

In a specific embodiment, a fusion protein comprising a mycobacterial hsp covalently linked to a peptide or protein is injected into a mammal, in which the fusion protein enters cells. For example, a fusion protein comprising a mycobacterial hsp and a moiety to be delivered into mammalian cells is injected as a soluble protein into a mammal (e.g., mouse, human) and the fusion protein enters the cells of the mammal. Thus, moieties such as whole proteins or peptides which typically do not enter cells efficiently, but which are functional entities once inside cells, are complexed to an hsp in order to efficiently introduce the moiety into cells. Similarly, chemicals which do not enter cells efficiently can be introduced into target cells by being complexed to hsps. Another example of the present invention is a fusion protein comprising an hsp and a functional molecule, such as a cellular protease, which is administered to a mammal and processed by cells of the mammal, thereby releasing a functional molecule (e.g., the protease) from the fusion once it enters the cell.

As used herein the term “heat shock protein” or “hsp”, also known as “stress protein”, is a protein which is synthesized in an organism in response to stresses to the organism, such as a rise in temperature and/or glucose deprivation. In particular embodiments, the hsp used in the methods of the present invention is an isolated (purified, essentially pure) hsp. The hsp can be isolated from the cell in which it occurs in nature using routine methods. In addition, the hsp can be produced using chemical or recombinant techniques (Maniatis et al., Molecular Cloning, A Laboratory Manual, 2nd ed., Cold Spring Harbor Laboratory Press, 1989). The term “hsp” also includes the entire hsp or a portion of the hsp of sufficient size to deliver or chaperone entry of a moiety into a cell. The term “hsp” also includes a protein having an amino acid sequence which is the functional equivalent of the hsp in that it is sufficiently homologous in amino acid sequence to that of the hsp to be capable of delivering or chaperoning entry into a cell of a moiety which does not enter cells on its own or enters cells on its own only to a limited extent. The term “sufficiently homologous in amino acid sequence to that of the hsp” means that the amino acid sequence of the protein or polypeptide will generally show at least 40% identity with the hsp amino acid sequence; in some cases, the amino acid sequence of a functional equivalent exhibits approximately 50% identity with the amino acid sequence of the hsp; and in some cases, the amino acid sequence of a functional equivalent exhibits approximately 75% identity with the amino acid sequence of the hsp. In a particular embodiment, the amino acid sequence of a functional equivalent exhibits approximately 95% identity with the amino acid sequence of the hsp.

Any suitable hsp can be used in the methods of the present invention. The hsp for use in the present invention can be, for example, a mycobacterial heat shock protein, a human heat shock protein, a yeast heat shock protein, a bacterial heat shock protein, a nonhuman mammalian heat shock protein, an insect heat shock protein or a fungal heat shock protein. In one embodiment, the heat shock protein is a mycobacterial heat shock protein such as hsp65, hsp70, hsp60, hsp71, hsp90, hsp100, hsp10-12, hsp20-30, hsp40 and hsp100-200.

The hsp can be conjugated or joined to any moiety which is not generally able to enter cells on its own or which enters cells on its own only to a limited extent. The moiety can be a protein, peptide, lipid, carbohydrate, glycoprotein and/or small organic molecule. In a particular embodiment, the moiety is a functional moiety. That is, the moiety has biological activity upon entry into the cell. For example, the moiety can be a functional enzyme, hormone, protease, toxin, toxoid and/or cytokine.

Since intact proteins in the extracellular medium do not ordinarily penetrate into a cell's cytosol, soluble proteins typically fail to stimulate mice to produce CTL (Braciale, T. J. et al., Immunol. Rev., 98:95-114 (1987)), although there are exceptions (Jondal, M. et al., Immunity, 5:295-302 (1996)). In comparison with other proteins, the soluble heat shock protein termed gp96 is an unusually effective stimulator of CD8 CTL (Udono, H. et al., Proc. Natl. Acad. Sci. USA, 91:3077-81 (1994)). Mice injected with gp96 isolated from tumor cells (donor cells) produce CTL that are specific for donor cell peptides in association with the responder mouse's class I MHC proteins (Udono, H. & Srivastava, P. K., J. Immunol., 152:5398-5403 (1994); Arnold, D. et al., J. Exp. Med., 182:885-889 (1995)). Since donor peptides are bound noncovalently by the isolated hsp protein, the results suggest that the hsp molecules are capable of delivering noncovalently associated peptides to MHC-1 proteins of other (recipient) cells, including antigen presenting cells.

The noncovalently bound peptide-gp96 complexes which are purified from a tumor cell appear to represent a broad array of proteins expressed by the cell (Arnold, D. et al., J. Exp. Med., 186:461-466 (1997); Li, Z. & Srivastava, P. K., Embo J, 12:3143-3151 (1993)). In contrast, recombinant hsp fusion proteins in which specific proteins of interest are covalently linked to the hsp provide a well-characterized polypeptide which lack extraneous peptides. In addition, a large protein fragment covalently linked to the hsp is an especially rich source of many different naturally processed peptides. Peptide mixtures of this kind, derived from specific antigens of interest, are particularly suitable for forming intracellular peptide-MHC complexes with the highly diverse MHC proteins found in different individuals of genetically outbred populations.

As described herein, a recombinant -hsp70 protein expression vector that permits diverse proteins and peptides to be fused to the amino terminus of mycobacterial -hsp70 was used to investigate whether soluble -hsp70 fusion proteins could be utilized to elicit MHC class-I restricted CD8⁺ CTL. Previously it has been shown that M. tuberculosis -hsp70 can be used as an adjuvant-free carrier to stimulate the humoral and cellular response to a full-length protein that is covalently linked to the hsp (Suzue, K. & Young, R. A., J. Immunol., 156:873-879 (1996)).

As demonstrated herein, a soluble -hsp70 fusion protein having a large fragment of chicken ovalbumin as fusion partner, in the absence of adjuvants, stimulates H-2^{b} mice to produce ovalbumin-specific CD8 CTL. The CTL recognized an immunodominant ovalbumin octapeptide, SIINFEKL (SEQ ID NO: 1), known to be a naturally processed peptide derived from ovalbumin expressed in mouse cells (Rotzschke, O. et al., Eur. J. Immunol., 21:2891-2894 (1991)), in the context of K^{b}. CTL from the immunized mice were as active cytolytically as a highly effective CTL clone (4G3) that had been raised against ovalbumin-expressing tumor cells, as both caused half-maximal lysis of K^{b+} target cells with the SIINFEKL peptide (SEQ ID NO: 1) at about the same concentration (10⁻¹³ M). The results indicate that the ovalbumin-hsp70 fusion protein, injected as a soluble protein into mice, can enter the MHC class I processing pathway in antigen presenting cells and stimulate the production of CD8 CTL.

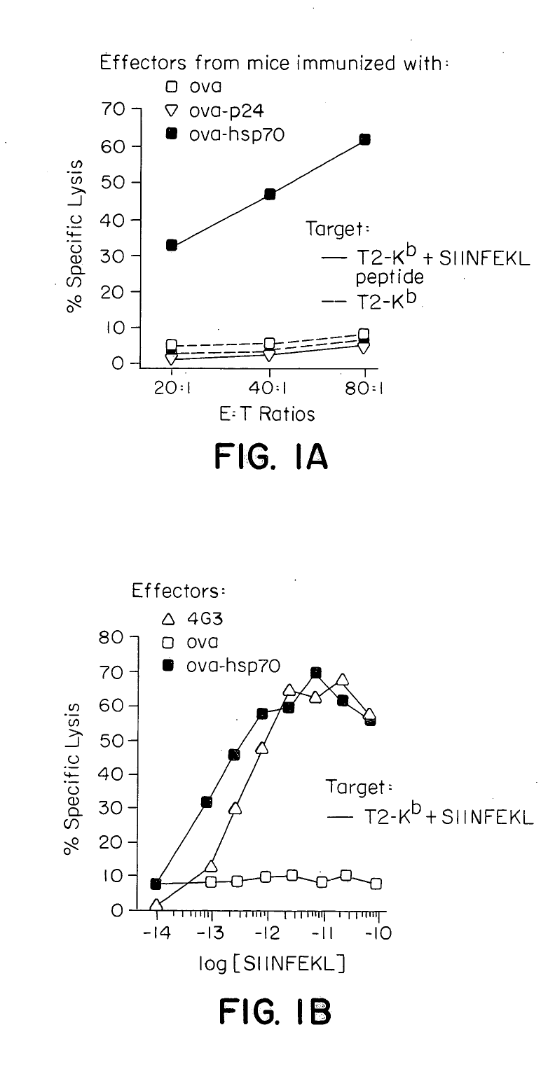

In particular, as described herein, injection of an -hsp70-ovalbumin fusion protein into H-2^{b} mice stimulated the production of CD8 CTL that recognize the immunodominant ovalbumin octapeptide, SIINFEKL (SEQ ID NO: 1), in association with K^{b}. The immunized mice were protected against an otherwise lethal challenge with an ovalbumin-expressing melanoma tumor, and their CTL were as effective (see FIG. 1B) in recognizing the SIINFEKL-K^{b} complex as a CTL clone (4G3) that was raised against cells (EG7-OVA) in which ovalbumin is expressed and processed naturally for class I-MHC presentation. These findings clearly imply that the covalently linked fusion partner of the injected hsp fusion protein was processed in the same way as ordinary cytosolic proteins for presentation with MHC class I proteins in antigen presenting cells.

Previously it was reported that mice injected with an HIV-1 gag protein (p24) linked to -hsp70 produced p24-specific T cells. Although the peptide-MHC complexes recognized by the T cells were not identified, the splenocytes from the fusion-protein immunized mice exhibited p24 antigen-dependent production of IFN-γ, which implies the presence of Th1 helper T cells and CTL. The previous findings, taken in conjunction with the present results, show that -hsp70 fusion proteins are generally useful as immunogens for stimulating CD8 CTL that are specific for peptides produced by natural proteolytic processing of the fusion partners within antigen presenting cells.

The mechanisms by which -hsp70 enables covalently linked polypeptide fusion partners to gain entry into the MHC class I processing pathway and elicit CD8 CTL could be based on: i) -hsp70's ability to assist protein folding (Zhu, X. et al., Science, 272:1606-1614 (1996), Flynn, G. C. et al., Nature, 353:726-730 (1991)), and to facilitate the translocation of proteins into subcellular compartments (Cyr, D. M. & Neupert, W., in Roles for -hsp70 in protein translocation across membranes of organelles, eds. Feige, U., Morimoto, R. I., Yahara, I. & Polia, B. S. (Birkhauser Veriag, Basel), Vol. 77, pp. 25-40 (1996); Brodsky, J. L., Trends. Biochem. Sci., 21:122-126 (1996)); ii) -hsp70's ability to facilitate the breakdown of intracellular proteins (Sherman, M. Y. & Goldberg, A. L., in Involvement of molecular chaperones in intracellular protein breakdown, eds. Feige, U., Morimoto, R. I., Yahara, I. & Polla, B. S. (Birkhauser Verlag, Basel), Vol. 77, pp. 57-78 (1996)); and iii) the high frequency of T cells directed against mycobacterial -hsp70.

-hsp70 is an integral component of the protein folding machinery (Hartl, F. U. et al., Trends Biochem. Sci., 19:20-25 (1994); Hartl, F. U., Nature, 381:571-579 (1996); Gething, M. J. & Sambrook, J., Nature, 355:33-45 (1992)) and functions through its ability to bind short linear peptide segments of folding intermediates. Detailed studies of the peptide-binding activity of -hsp70 have shown that it has a clear preference for peptides with aliphatic hydrophobic side chains (Flynn, G. C. et al., Nature, 353:726-730 (1991); Rudiger, S. et al., Embo. J., 16:1501-1507 (1997)). Thus -hsp70 appears to transiently associate with hydrophobic protein regions and prevent protein aggregation. The kinetics of -hsp70-substrate binding is governed by the ATP binding and ATPase activity of -hsp70 (Flynn, G. C. et al., Science, 245:385-390 (1989)). The combination of the peptide and ATP binding functions of -hsp70 may be involved in the efficient transfer of antigenic peptides into the MHC class I antigen presentation pathway. -hsp70 also associates with nascent polypeptide chains as they emerge from ribosomes and are involved in stabilizing nascent polypeptides prior to their translocation into various subcellular compartments (Beckmann, R. P. et al., Science, 248:850-854 (1990); Frydman, J. et al., Nature, 370:111-117 (1994)), including chloroplasts, the ER, lysosomes, mitochondria, the nucleus and peroxisomes (Cyr, D. M. & Neupert, W., Roles for -hsp70 in protein translocation across membranes of organelles, eds. Feige, U., Morimoto, R. I., Yahara, I. & Polia, B. S. (Birkhauser Veriag, Basel), Vol. 77, pp. 25-40 (1996); Brodsky, J. L., Trends. Biochem. Sci., 21:122-126 (1996)). The present findings indicate that -hsp70 also promotes delivery of covalently linked fusion polypeptides to the subcellular compartment(s) required for cell surface presentation of peptide-MHC-1 complexes.

-hsp70's role in intracellular protein breakdown may be especially relevant for the immunogenic effectiveness of its fusion partner. Experiments with yeast cell mutants and with mammalian cell extracts have shown that, in addition to its function in protein refolding, -hsp70 serves an essential role in the degradation of certain abnormal polypeptides (Sherman, M. Y. & Goldberg, A. L., Involvement of molecular chaperones in intracellular protein breakdown, eds. Feige, U., Morimoto, R. I., Yahara, I. & Polla, B. S. (Birkhauser Verlag, Basel), Vol. 77, pp. 57-78 (1996); Nelson, R. J. et al., Cell, 71:97-105 (1992)). Thus, if -hsp70 fails to refold a denatured protein, it can facilitate its degradation by the cell's proteolytic machinery. In eukaryotes, -hsp70 is essential for the ubiquitination of certain abnormal and regulatory proteins and thus in the breakdown of polyubiquinated polypeptides by the 26S proteasome (Sherman, M. Y. & Goldberg, A. L., Involvement of molecular chaperones in intracellular protein breakdown, eds. Feige, U., Morimoto, R. I., Yahara, I. & Polla, B. S. (Birkhauser Verlag, Basel), Vol. 77, pp. 57-78 (1996)). The peptides generated by the proteasome in the cytosol appear to be the primary source of the peptides that are translocated into the ER for association with MHC class 1. Thus proteins that are linked to -hsp70 may be ubiquitinated and processed especially well for presentation with MHC-1 proteins.

Immune responses to -hsp70 have been detected following exposure to a broad spectrum of infectious agents (Selkirk, M. E. et al., J. Immunol., 143:299-308 (1989); Hedstrom, R. et al., J. Exp. Med., 165:1430-1435 (1987); Young, D. et al., Proc. Natl. Acad. Sci. USA, 85:4267-4270 (1988)). In addition, anti-hsp70 immune responses were induced in infants by the trivalent vaccine against tetanus, diphtheria and pertussis (Del Giudice, G. et al., J. Immunol., 150:2025-2032 (1993)). It seems that the immune system is routinely stimulated to respond to -hsp70 and such stimulation may cause an expansion of -hsp70-reactive cells. The cellular responses to mycobacterial hsps are profound; limiting dilution analysis indicates that 20% of the murine CD4⁺ T lymphocytes that recognize mycobacterial antigens are directed against hsp60 alone (Kaufmann, S. H. et al., Eur. J. Immunol., 17:351-357. (1987)). The high frequency with which human CD4⁺ T cell clones directed against mycobacterial -hsp70 and hsp60 have been detected suggests that these hsps are also major targets of the cellular response in humans (Munk, M. E. et al., Eur. J. Immunol., 18:1835-1838 (1988)). Thus, although soluble proteins administered in the absence of adjuvant do not typically elicit CD8 CTL, it is likely that the abundant -hsp70 -reactive helper T cells are involved in facilitating the unusually efficient CTL response against the soluble -hsp70 fusion protein.

Another hsp, gp96, isolated from various tumors and tumor cell lines, has previously been shown to be a potent immunogen for eliciting CD8 CTL. Gp96's effectiveness derives from i) the many peptides that remain bound noncovalently to the protein when isolated from cells (Arnold, D. et al., J. Exp. Med., 186:461-466 (1997); Li, Z. & Srivastava, P. K. Embo. J., 12:3143-3151 (1993)); and ii) its ability to facilitate the transfer of those peptides to MHC-1 proteins of “professional” antigen presenting cells (Suto, R. & Srivastava, P. K., Science, 269:1585-1588 (1995)). Detailed studies of the peptide-binding activity of -hsp70 has shown that it has a clear preference for peptides over 7 amino acids in length and those with aliphatic hydrophobic side chains (Flynn, G. C. et al., Nature, 353:726-730 (1991); Rudiger, S. et al., Embo. J., 16:1501-1507 (1997)). Although gp96 can bind many different peptides (Arnold, D. et al., J. Exp. Med., 182:885-889 (1995); Udono, H. & Srivastava, P. K., J. Exp. Med., 178:1391-1396 (1993); Nieland, T. J. et al., Proc. Natl. Acad. Sci. USA, 93:6135-6139 (1996)), studies with -hsp70, as well as general considerations, indicate that no protein can serve as a universal receptor for all peptides. Recombinant -hsp70 fusion proteins, in contrast, are thus likely to provide a richer source of peptides available for binding to diverse MHC molecules.

Many different proteins can be linked to -hsp70 and the fusion proteins studied so far are effective immunogens in the absence of adjuvants. -hsp70 fusion proteins are thus attractive candidates for vaccines intended to stimulate CD8 CTL in humans.

As also described herein, the ability of hsp fusion vaccines to elicit MHC class I-restricted CTLs against the attached protein moiety indicates that the fusion protein is able to enter cells, as an intact molecule, and find its way into the class I antigen presentation pathway. Antigens such as ovalbumin cannot elicit a CTL response without being fused to hsp70, indicating that the heat shock protein is necessary for cellular entry. This ability of hsps to enter cells can be used to deliver molecules that normally cannot enter cells on their own. For example, whole proteins or peptides which typically do not enter cells efficiently, but which have functional capacities once inside cells, could be fused to a heat shock protein in order to efficiently introduce them into cells. Similarly, chemicals which do not enter cells efficiently can be introduced into target cells by being fused to hsps. If necessary, the fusion protein can be engineered to become digested with a cellular protease to release a functional molecule from the fusion once it enters the cell.

Thus, the methods of the present invention can be used therapeutically or diagnostically to deliver a moiety (one or more), which is not generally able to enter cells or which enters cells only to a limited extent, into cells or into cells of an individual. In addition, the methods of the present invention can be used to deliver a moiety to a tissue or organ (e.g., of an individual). In a particular embodiment, the cells, tissues or organs are mammalian (e.g., murine, canine, feline, bovine, monkey and human) cells, tissues or organs.

In the method of the present invention wherein a moiety is delivered into mammalian cells, tissues or organs, for therapeutic purposes, an effective amount of the complex comprising the moiety of interest linked to a hsp is administered to the mammalian cell, tissue or organ. An “effective amount” is an amount such that when administered, it results in delivery of the complex comprising the moiety linked to the hsp into the cell, tissue or organ. In addition, the amount of the complex used to deliver a moiety into a cell, tissue or organ will vary depending on a variety of factors, including the moiety being delivered, the size, age, body weight, general health, sex and diet of the individual, and the time of administration, duration or particular qualities of the condition being treated therapeutically.

Various delivery systems can be used to administer the complex to cells, tissues or organs. Methods of introduction include, for example, subcutaneous, intramuscular, intraperitoneal, intravenous, intradermal, intranasal, epidural and oral routes. Any other convenient route of administration can be used (infusion of a bolus injection, infusion of multiple injections over time, absorption through epithelial or mucocutaneous linings such as oral, mucosa, rectal or intestinal mucosa).

The following Examples are offered for the purpose of illustrating the present invention and are not to be construed to limit the scope of this invention. The teachings of all references cited herein are hereby incorporated herein by reference.

### EXEMPLIFICATION

Materials and Methods

Expression Vector Constructs

The DNA fragment containing the M. tuberculosis -hsp70 coding sequence was synthesized by PCR using DNA purified from λgt11 clones Y3111 and Y3130 as a template (Young, D. B., Kent, L. & Young, R. A., Infect. Immun., 55:1421-1425 (1987)). The complete coding sequence of -hsp70 was synthesized by using the upstream primer oKS63 (5′GCCCGGGATCCATGGCTCGTGCGGTCGGGAT3′) (SEQ ID NO: 3) containing a BamHI site immediately before the -hsp70 coding sequence and the downstream primer oKS79

(5′GCGGAATTCTCATCAGCCGAGCCGGGGT3′) (SEQ ID NO: 4) containing an EcoRI site immediately after the last coding sequence of -hsp70. The DNA fragment containing the ovalbumin coding sequence was synthesized by PCR using plasmid pOv230 (McReynolds, L. et al., Nature, 273:723-728 (1978)) as a template. The upstream primer oKS83

(5′GCGGATCCATATGGTCCTTCAGCCAAGCTCCGTGG3′) (SEQ ID NO: 5) contained a NdeI site immediately before amino acid 161 of ovalbumin and the downstream primer oKS82 (5′GCAGGATCCCTCTTCCATAACATTAGA3′) (SEQ ID NO: 6) contained a BamHI site immediately after amino acid 276 of ovalbumin. Another downstream primer containing a BamHI site oKS80

(5′GCTGAATTCTTACTCTTCCATAACATTAG3′) (SEQ ID NO: 7), included a translation stop codon immediately after amino acid 276 of ovalbumin.

Construction of the vector used to produce -hsp70 alone, pKS74, has been previously described (Suzue, K. & Young, R. A., J. Immunol., 156:873-879 (1996)). The vector pKS11h was made by modifying the plasmid vector pET11 (Studier, F. W. et al., Methods Enzymol., 185:60-89 (1990)) with a histidine tag coding sequence and with the polylinker from pET17b. Plasmid pKS28 was made by subcloning the DNA encoding amino acids 161 to 276 of ovalbumin into the NdeI and BamHI sites of pKS11h. Plasmid pKS76 was created by subcloning ovalbumin (161-276) and -hsp70 into the NdeI and BamHI sites of pKS11h.

Protein Purification

Cultures of BL21(DE3)pLysS (Studier, F. W. et al., Methods Enzymol., 185:60-89 (1990)) were grown and induced with 0.5 mM isopropylthiogalactoside (IPTG). -hsp70 and ova-hsp70 proteins were both purified as inclusion bodies, refolded stepwise in guanidine and subsequently purified by ATP affinity chromatography as previously described (Suzue, K. & Young, R. A., J. Immunol., 156:873-879 (1996)). Protein purity was verified by SDS-PAGE and protein fractions were pooled and dialyzed against PBS. Protein concentrations were determined by the bicinchoninic acid assay (Pierce, Rockford, Ill.).

Peptides

The peptides SIINFEKL (corresponding to ovalbumin amino acids 258-276) (SEQ ID NO: 1) and RGYVYQGL (corresponding to the vesicular stomatitis virus nucleoprotein amino acids 324-332) (SEQ ID NO: 2), were synthesized by the Biopolymers Facility at the Center for Cancer Research at the Massachusetts Institute of Technology. Peptides were stored as 1 mg/ml stock solutions in PBS.

Mice and Immunizations

Seven-eight week old female C57BL/6 mice were obtained from Jackson Laboratories (Bar Harbor, Me.) and Taconic Farms (Germantown, N.Y.). Mice were immunized i.p. on day 0 and s.c. on day 14 with 120 pmoles of purified protein in PBS.

Cell lines

EL4 (H-2^{b}) thymoma cells, from the American Type Culture Collection (ATCC, Rockville, Md.), were grown in RPMI 1640/10% FCS. E.G7-OVA cells (ovalbumin transfected EL4 cells) (Moore, M. W. et al., Cell, 54:777-785 (1988)) were cultured in RPMI 1640/10% FCS in the presence of 320 μg of G418 per ml. The human cell line T2, is a TAP-deficient, T-B lymphoblastoid fusion hybrid. The K^{b} transfected clone, T2-K^{b}, a generous gift from P. Cresswell, was cultured in RPMI 1640/10% FCS in the presence of 320 μg of G418 per ml. The CTL clone 4G3 was maintained by weekly restimulation with irradiated E.G7-OVA cells in RPMI 1640/10% FCS/5% rat Con A supernatant (Walden, P. R. & Eisen, H. N., Proc. Natl. Acad. Sci. USA, 87:9015-9019 (1990)). The C57BL/6-derived melanoma B16 and the ovalbumin-transfected B16 clone, M05, (Falo, L., Jr., et al., Nat. Med., 1:649-653 (1995)) were generously provided by L. Rothstein and L. Sigal. The B16 cells were grown in RPMI 1640/10% FCS and the M05 cells were grown in the presence of 2.0 mg of G418 and 40 μg of hygromycin per ml.

IFN-γ ELISA

Spleens were removed from mice 10 days after the last injection. The spleens from 3-10 mice in each treatment group were pooled. Single-cell suspensions were prepared by grinding tissue through a sterile nylon mesh. Erythrocytes were removed by suspending the cells in pH 7.2 lysis buffer (0.15 M NH₄Cl, 1 M KHCO₃, 0.1 mM Na₂EDTA) and rinsing the cells two times with RPMI 1640 media. Splenocytes were then cultured at 1×10 cells/ml in 96-well round bottom microculture plates in RPMI 1640, supplemented with 10% FCS and 50 μM 2-ME at 37° C. in 5% CO². The cells were stimulated with recombinant ovalbumin (10 μg/ml), SIINFEKL peptide (SEQ ID NO: 1) (10 μg/ml), RGYVYQGL (SEQ ID NO: 2) (10 μg/ml) or with Con A (5 μg/ml). Cell culture supernatants were removed at 72 h. A sandwich ELISA using paired monoclonal antibodies (Endogen, Cambridge, Me.) was used to measure IFN-γ.

CTL assay

Single-cell suspensions of splenocytes were prepared as above. 25×10⁶ splenocytes were cultured with 5×10⁶ irradiated (15,000 rads) E.G7-OVA cells in RPMI 1640 supplemented with 10% FCS, 50 μM 2-ME, 1 mM sodium pyruvate and 100 μM non-essential amino acids. After 6-7 days in culture, splenocytes were purified by Ficoll-Paque (Pharmacia, Piscataway, N.J.) density centrifugation and then utilized as effector cells.

Target cells were labeled with 100 μCi[⁵¹Cr] at 37° C. for 1-2 h. For peptide sensitization of target cells, 50 μg of peptide was added to the target cells (300 μg/ml final peptide concentration) during the labeling period. The cells were then rinsed and 5000 [⁵¹Cr]-labeled targets and serial dilutions of effector cells were incubated at various E:T ratios in 96 well U-bottom plates at 37° C. For peptide titration assays, the target cells were not pulsed with any peptide during the [⁵¹Cr]-labeling period and instead, the peptide was directly added to the 96 well U-bottom plate at final concentrations of 10⁻¹⁰ M to 10⁻¹⁴ M. Supernatants were harvested after 4-6 h and the radioactivity was measured in a gamma counter. % Specific lysis was calculated as equal to 100 X [(release by CTL-spontaneous release)/(maximal release-spontaneous release)]. Maximal release was determined by addition of 1% Triton X-100 or by resuspending target cells.

In vitro depletion or enrichment of lymphocyte subpopulations

Splenocytes were cultured with irradiated E.G7-OVA cells and purified by Ficoll-Paque (Pharmacia) density centrifugation as described above. Cells were resuspended in cold PBS with 1% FCS and incubated with anti-mouse CD4 (L3T4) microbeads or with anti-mouse CD8a (Ly-2) microbeads (Miltenyi Biotech, Bergisch Gladbach, Germany) for 20 min. at 4° C. For cell depletion, the cells were applied on to a Mini MACS column (Miltenyi Biotech) with an attached flow resistor. The cells from the flow-through were collected and used as effector cell in the cytolytic assay. For positive selection of CD8 cells, the cells were applied on to a Mini MACS column without a flow resistor. The column was rinsed and the cells adhering to the column were released by removing the column from the magnetic holder. The positively selected cells were then used as effector cells in the cytolytic assay. The effectiveness of positive and negative selection of cells was verified by flow cytometry using phycoerythrin conjugated anti-mouse CD4 and fluorescein isothiocyanate conjugated anti-mouse CD8a antibodies (Pharmingen, San Diego, Calif.).

Tumor protection assay

C57BL/6 mice were injected i.p. with 120 pmoles of ova or ova-hsp70 without adjuvant and boosted s.c. 2 weeks later. Ten days after the last immunization the mice were injected s.c. on the right flank with 1×10⁵ MO5 tumor cells or with 1×10⁵ B16 tumor cells. As a control, unimmunized mice were also inoculated with the tumor cells. Five to ten mice were used for each experimental group. On the day of the tumor challenge, the B16 and MO5 cells were harvested by trypsinization and rinsed three times in PBS. The cells were resuspended in PBS and administered s.c. in a volume of 0.1 ml. Tumor growth was assessed by measuring the diameter of the tumor in millimeters (recorded as the average of two perpendicular diameter measurements). Mice that became moribund were sacrificed. Consistent results were observed in three separate experiments.

### RESULTS

Purified recombinant proteins

A recombinant system developed to permit production of M. tuberculosis -hsp70 fusion proteins in E. coli (Suzue, K. & Young, R. A., J. Immunol., 156:873-879 (1996)) was utilized to attach amino acids 161 to 276 of ovalbumin to the N-terminus of M. tuberculosis -hsp70 . A comparable recombinant ovalbumin protein (amino acids 161 to 276) was also produced. The selected portion of ovalbumin contains the immunodominant epitope SIINFEKL (SEQ ID NO: 1) recognized by CTL in association with K^{b} (Rotzschke, O. et al., Eur. J. Immunol., 21:2891-2894 (1991); Carbone, F. R. & Bevan, M. J., J. Exp. Med., 169:603-612 (1989)). The ovalbumin -hsp70 fusion protein and the ovalbumin (aa 161-276) protein were expressed at high levels in E. coli. These proteins were purified as inclusion bodies, refolded in vitro, and further purified by column chromatography. The purity of the recombinant proteins was assessed by SDS-PAGE. E. coli cell lysates and purified proteins were examined by SDS-PAGE and proteins were visualized by Coomassie staining. The gel contained crude extracts from IPTG-induced E. coli containing pKS28 (ova 161-276) and from IPTG-induced E. coli containing pKS76 (ova-hsp70 ), and the purified proteins ova 161-276 and ova-hsp70. Examination of commercial preparations of crystallized and high grade ovalbumin by SDS-PAGE and silver staining revealed that they are highly contaminated with low molecular weight polypeptides. For this reason, only the highly purified recombinant ovalbumin (aa 161-276) protein, referred to below simply as ovalbumin, was used in these studies.

Immunization of mice with -hsp70 fusion protein in PBS elicits T cell responses against the attached antigen

Whether mice injected with soluble protein without adjuvant could be primed to produce anti-ovalbumin T cells was investigated (FIG. 1A). C57BL/6 mice were inoculated i.p. with 120 pmoles of ovalbumin (ova) or with 120 pmoles of ovalbumin-hsp70 fusion protein (ova-hsp70 ) in PBS. A second equivalent dose was given s.c. at two weeks. A third group of mice was injected with 120 pmoles of ovalbumin-p24 gag fusion protein (ova-p24), purified as described in (Suzue, K. & Young, R. A., J. Immunol., 156:873-879 (1996)), in order to examine the immune responses elicited by administering ovalbumin covalently linked to a protein other than -hsp70, in the absence of adjuvant. Splenocytes of immunized mice were removed ten days after the s.c. immunization and for each mouse group, 5-10 spleens were pooled and splenocytes from immunized mice were cultured in vitro for 6 days with irradiated E.G7-OVA cells (syngeneic EL4 cells transfected with ovalbumin) without added interleukins (Moore, M. W. et al., Cell, 54:777-785 (1988)). The cultured cells were then used as effector cells in CTL assays. Cells from mice injected with ovalbumin protein or with ovalbumin-p24 fusion protein were unable to lyse T2-K^{b} target cells or T2-K^{b} cells pulsed with SIINFEKL peptide (SEQ ID NO: 1). In contrast, effector cells from mice primed with ovalbumin-hsp70 fusion protein were able to lyse T2-K^{b} cells pulsed with SIINFEKL peptide (SEQ ID NO: 1). See FIG. 1A wherein the splenocyte cultures derived from mice immunized with ova □, ova-p24 ∇ and ova-hsp70 ▪, which were used as effector cells in a standard cytotoxicity assay, is shown. The following ⁵¹Cr-labeled target cells were used: T2-K^{b} cells_{——} and T2-K^{b} pulsed with SIINFEKL peptide_{—} at 300 μg/ml.

Results obtained with other target cells also show that the anti-ovalbumin CTL recognized SIINFEKL (SEQ ID NO: 1) in association with K^{b}. Splenocytes from ovalbumin-hsp70 immunized mice were able to lyse both E.G7-OVA target cells and EL4 cells pulsed with SIINFEKL (SEQ ID NO: 1) peptide but were unable to lyse EL4 cells in the absence of peptide or EL4 cells pulsed with another K^{b}-binding peptide (RGYVYQGL (SEQ ID NO: 2), from vesicular stomatitis virus, (Van Bleek, G. M. & Nathenson, S. G., Nature, 348:213-216 (1990)).

To assess the effectiveness of the CTL from ova-hsp70-immunized mice, they were tested after 6 days in culture in cytolytic assays using T2-K^{b} as target cells and SIINFEKL (SEQ ID NO: 1) at various concentrations. For purposes of comparison, the assay included a well-characterized CTL clone (4G3) that recognizes the SIINFEKL-K^{b} complex. As shown in FIG. 1B, half-maximal lysis was obtained with both the CTL line and the 4G3 clone at about the same peptide concentration, approximately 5×10⁻¹³ M. Thus CTL from the ova-hsp70 immunized mice and the clone against the ovalbumin-expressing tumor (E.G7-OVA) were equally effective in terms of the SIINFEKL (SEQ ID NO: 1) concentration required for half-maximal lysis. It may be noted that in FIG. 1B the ratio of 4G3 cells to target cells (E:T ratio) was 5:1, whereas for the CTL line this ratio was 80:1. While the E:T ratio has a large impact on the maximal lysis of target cells at 4 hr, changing this ratio over an 80-fold range (1:1 to 80:1) has a negligible effect on the peptide concentration required for half-maximal lysis.

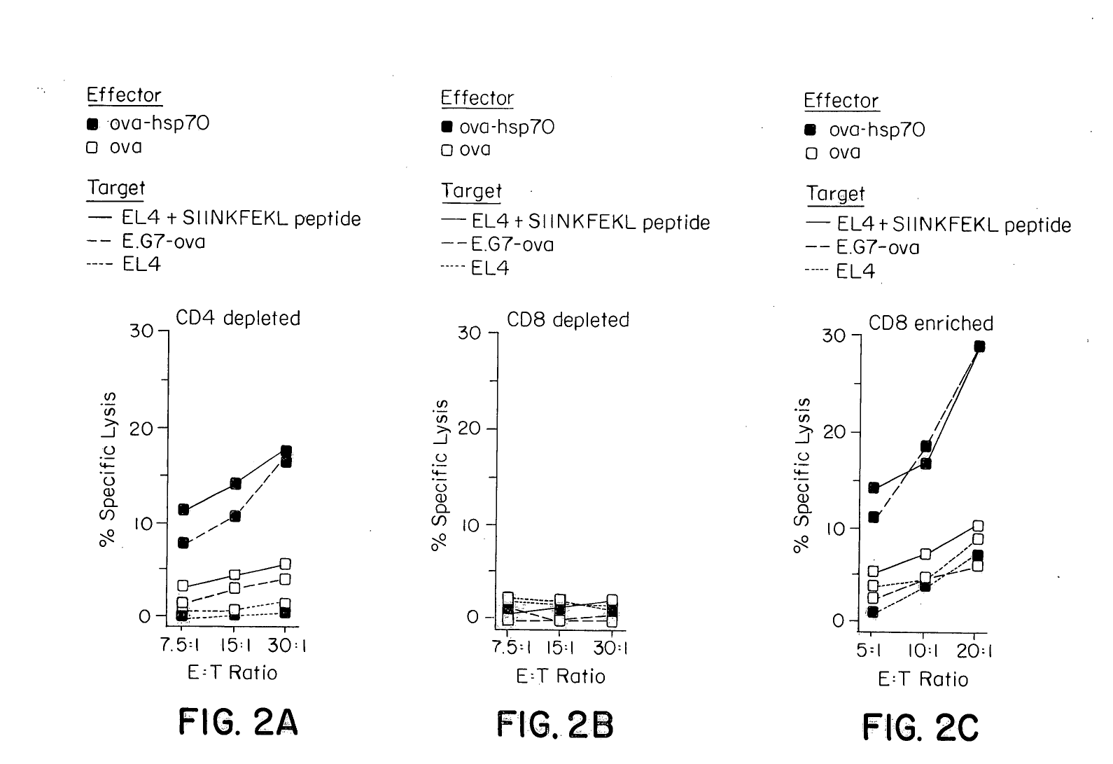

Next, that the cytolytic activity of the CTL line from ova-hsp70 immunized mice was due to CD8⁺ T cells was verified (FIGS. 2A-2C). C57BL/6 mice were injected i.p. with 120 pmoles of ova or ova-hsp70 without adjuvant and boosted s.c. with the same amounts of these proteins 2 weeks later. Mice were sacrificed 10 days after the boost and for each mouse group, 5-10 spleens were pooled and splenocytes were incubated for 6 days in the presence of irradiated E.G7-OVA cells. Prior to performing the cytotoxicity assay, the effector cells were negatively or positively selected for CD4⁺ cells or CD8⁺ cells using paramagnetic antibodies (see Materials and Methods). Splenocyte cultures were either depleted of CD4⁺ cells (CD4-CD8⁺) (FIG. 2A), depleted of CD8⁺ cells (CD4⁺ CD8⁻) (FIG. 2B) or were enriched for CD8⁺ cells (CD8⁺) (FIG. 2C). A MACS column to separate the CTL line into T cell subsets (see Materials and Methods) was used. CTL activity was unaffected by removing CD4⁺ cells, but it was completely abrogated by removing CD8⁺ cells. Retrieval of the CD8⁺ cells from the MACS column led to recovery of cytolytic activity. The results were the same when target cells were EL4 cells incubated with SIINFEKL (SEQ ID NO: 1) or ovalbumin expressing EL4 cells (E.G7-OVA). Thus, administration of ovalbumin-hsp70 fusion protein, but not ovalbumin alone, elicits CD8⁺ CTL specific for SIINFEKLK^{b} (SEQ ID NO: 1).

The lower level of cytolytic activity in FIGS. 2A-2C relative to FIG. 1A and FIG. 1B reflects the different target cells used. T2-K^{b} cells (FIGS. 1A-1B) and EL4 cells (FIGS. 2A-2C) have approximately the same high level of cell surface K^{b} (roughly 100,000 molecules per cell), but the peptide transporter (TAP) is defective in T2-K^{b} (Anderson, K. S. et al., J. Immunol., 151:3407-3419 (1993)), and not in EL4. Hence, at a given free concentration of SIINFEKL (SEQ ID NO: 1) the target cell epitope density (number of SIINFEKL K^{b} complexes per cell) is much greater on T2-K^{b} than EL4 cells.

Hsp70 must be covalently coupled to ovalbumin to engender antiovalbumin T cell responses

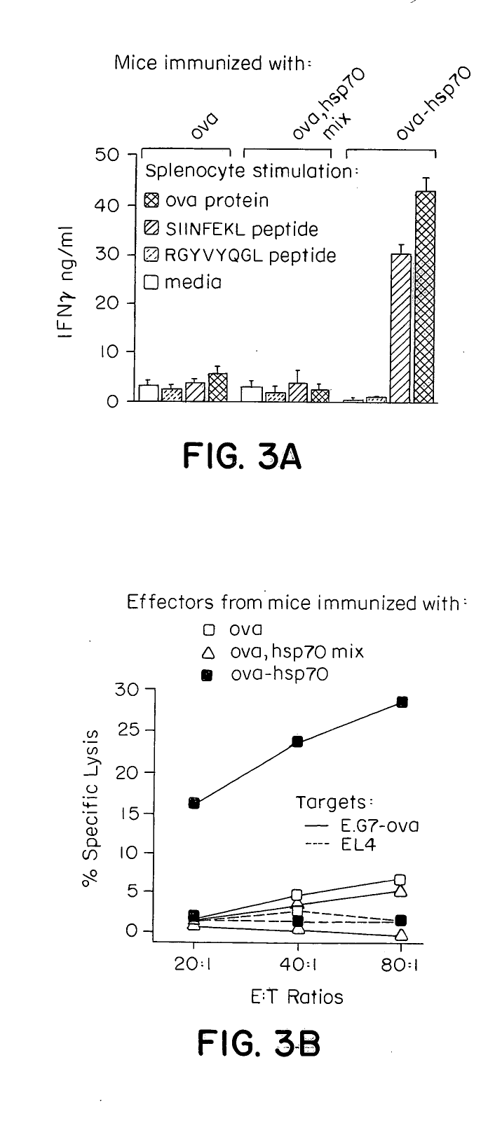

Next, it was examined whether the covalent fusion of -hsp70 to ovalbumin was necessary to elicit cellular responses to ovalbumin or whether the same results could be obtained if the two proteins were simply mixed but not covalently attached (FIGS. 3A-3B). Mice were injected with 120 pmoles of ovalbumin-hsp70 fusion protein, with 120 pmoles of ovalbumin, or with 120 pmoles of -hsp70 mixed with 120 pmoles of ovalbumin. Ten days after the boost 5-10 spleens from each mouse group were pooled and processed. The level of IFN-γ secreted by the splenocytes in response to restimulation with ovalbumin in vitro was measured by ELISA. Splenocytes from mice immunized with ovalbumin alone or with a mixture of ovalbumin and -hsp70 proteins produced less than 6 ng/ml IFN-γ in response to stimulation with SIINFEKL peptide (SEQ ID NO: 1) or ovalbumin (FIG. 3A). In contrast, splenocytes from mice injected with the ovalbumin-hsp70 fusion protein secreted substantially higher levels of IFN-γ when restimulated in vitro with SIINFEKL peptide (SEQ ID NO: 1) or ovalbumin. The release of IFN-γ was ovalbumin specific, since splenocytes cultured in media alone or with control RGYVYQGL peptide (SEQ ID NO: 7) secreted low levels of IFN-γ.

Similar results were obtained by cytolytic assays. See FIG. 3B wherein splenocyte cultures from mice immunized with recombinant ova □, ova-hsp70 fusion protein ▪ or with a mixture of ova and -hsp70 proteins Δ, were used as effector cells in a standard cytotoxicity assay is shown. The following ⁵¹Cr-labeled target cells were used: E.G7-OVA_{—} and EL4 cells alone_{——}. Ovalbumin-specific CTL were produced by mice injected with the ovalbumin-hsp70 fusion protein but not by those injected with a mixture of ovalbumin with -hsp70.

Immunization of mice with ovalbumin-hsp70 protein without adjuvant engenders protective immunity to M05 tumor challenge

The MO5 cell line, which is a B16 melanoma cell line transfected with ovalbumin expressing DNA, presents the immunodominant SIINFEKL peptide (SEQ ID NO: 1) in association with K^{b} on the cell surface (Falo, L., Jr., et al., Nat. Med., 1:649-653 (1995)). Using this tumor it was determine whether the immune response induced by ovalbumin-hsp70 fusion protein is sufficient to engender protective tumor immunity. Mice were injected i.p. with 120 pmoles of ovalbumin or ovalbumin-hsp70 without adjuvant and boosted s.c. 2 weeks later. Ten days later the mice were injected s.c. on the right flank with 1×10⁵ tumor cells or with 1×10⁵ B16 tumor cells. As an additional control, naive mice were also inoculated with the tumor cells.

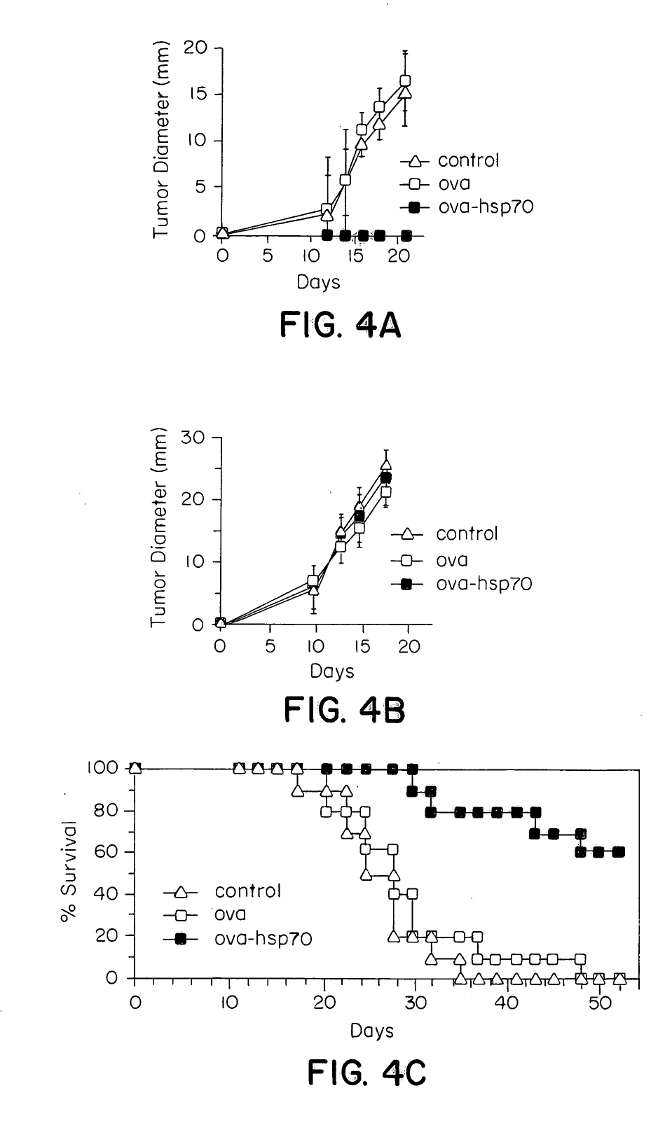

All mice challenged with tumor cells were monitored for tumor growth and growth was recorded as the average tumor diameter in millimeters (FIGS. 4A-4B). Twenty-one days following the MO5 tumor challenge, the average tumor diameter in the control and the ovalbumin immunized mice was greater than 15 mm. Because the control and ovalbumin immunized mice began dying 21 days after the tumor challenge, tumor growth was not recorded beyond 21 days. In contrast to the control and the ovalbumin-immunized mice, no tumors were detected in the ovalbumin-hsp70 immunized mice 21 days after the tumor challenge. All groups of mice (control, ovalbumin-immunized or ovalbumin--hsp70 immunized) which were challenged with the B16 tumor cells developed tumors and became moribund by 21 days after the tumor challenge.

The survival of mice was recorded as the percentage of mice surviving following the tumor challenge (FIG. 4C). Mice which appeared moribund were sacrificed. Forty days after the M05 tumor challenge, none of the control mice and only 10% of the ovalbumin-immunized mice had survived. In contrast, 80% of the ovalbumin-hsp70 immunized mice had survived. These experiments demonstrate that immunization of mice with the ovalbumin-hsp70 fusion protein, but not with the ovalbumin protein alone, induces ovalbumin specific protective tumor immunity.

Administering Ovalbumin-hsp70 Fusion Protein Containing either the ATP Binding or the Peptide Binding Domain of hsp70 is Sufficient to Elicit anti-ovalbumin T Cell Responses

Whether the peptide binding or the ATP binding domain of hsp70 was sufficient for eliciting T cell responses to the attached ovalbumin antigen was investigated. It is possible that since the ATPase and ATP binding functions of the hsp70 protein were not essential for its adjuvant-free carrier function, that the presence of this function domain of hsp70 is unnecessary when utilizing the ovalbumin-hsp70 fusion protein to elicit anti-ovalbumin T cell responses. The amino terminal 44 kD portion of hsp70 has been characterized as the ATP binding domain with ATPase activity and the carboxyl terminal portion of hsp70 binds polypeptide substrates. Recombinant fusion proteins were produced with the ATP binding domain of hsp70 attached to ovalbumin (ovalbumin-NH₂ hsp70) and the peptide binding domain of hsp70 attached to ovalbumin (ovalbumin CO₂H hsp70). These proteins were purified from E. coli as inclusion bodies, refolded and purified using NTA-Ni²⁺ chromatography.

The T cell responses to ovalbumin were assessed after injecting mice with ovalbumin-NH₂ hsp70 or with ovalbumin-CO₂ hsp 70 fusion protein in saline solution. Levels of IFNg secreted by the splenocytes in response to OVA8 peptide was 22 ng/ml in the ovalbumin-NH₂ hsp70 group and was 19 ng/ml in the ovalbumin-CO₂H hsp70 group. When splenocytes were stimulated with the ovalbumin protein antigen, the IFNg levels were 38 ng/ml in the ovalbumin-NH₂ hsp70 group and was 29 ng/ml in the ovalbumin-CO₂H hsp70 group. In the cytolytic assay, the effector cells from both of these groups were able to effectively lyse OVA8 pulsed EL4 target cells and E.G7-OVA target cells but not the EL4 control cells. Administering soluble protein with either the amino or the carboxyl terminal portion of hsp70 fused to ovalbumin is sufficient to elicit anti-ovalbumin T cell responses.

DISCUSSION

Mice immunized with heat shock proteins (hsp) isolated from mouse tumor cells (donor cells) produced CD8 cytotoxic T lymphocytes (CTL) that recognized donor cell peptides in association with the MHC class I proteins of the responding mouse. The CTL are likely induced because peptides noncovalently associated with the isolated hsp molecules can enter the MHC class I antigen processing pathway of professional antigen presenting cells. Using a recombinant heat shock fusion protein with a large fragment of ovalbumin covalently linked to mycobacterial -hsp70, it has been shown herein that when the soluble fusion protein was injected without adjuvant into H-2^{b} mice, CTL were produced that recognized an ovalbumin-derived peptide, SIINFEKL (SEQ ID NO: 1), in association with K^{b}. The peptide is known to arise from natural processing of ovalbumin in H-2^{b} mouse cells, and both CTL from the ova-hsp70 -immunized mice and a highly effective CTL clone (4G3) raised against ovalbumin-expressing EL4 tumor cells (EG7-OVA), were equally effective in terms of the concentration of SIINFEKL (SEQ ID NO: 1) required for half-maximal lysis in a CTL assay. The mice were also protected against lethal challenge with ovalbumin-expressing melanoma tumor cells. Because large protein fragments or whole proteins serving as fusion partners can be cleaved into short peptides in the MHC class I processing pathway, hsp fusion proteins of the type described herein can be used to deliver moieties or molecules (e.g., proteins, peptides, lipids) which are not generally able to enter cells or enter cells only to a limited extent, into cells.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of delivering a moiety of interest into a cell, the method comprising:
(a) providing a complex comprising the moiety of interest covalently linked to the amino terminal ATP binding domain of a heat shock protein 70 (hsp70) or the carboxyl terminal peptide binding domain of an hsp70 and
(b) contacting the cell with the complex under conditions appropriate for entry of the complex into the cell.

2. The method of claim 1, wherein the hsp70 is a mycobacterial hsp70.

3. The method of claim 1, wherein the moiety of interest is a protein or a peptide.

4. The method of claim 1, wherein the cell is an antigen presenting cell.

5. The method of claim 3, wherein the protein or peptide is glycosylated.

6. The method of claim 1, wherein the moiety comprises a lipid, carbohydrate, or small organic molecule.

7. The method of claim 1, wherein the cell is a human cell.

8. The method of claim 1, wherein the moiety of interest is covalently linked to the hsp70 by a peptide bond.

9. The method of claim 1, wherein the moiety of interest is covalently linked to the hsp70 by chemical conjugation.

10. The method of claim 1, wherein the cell is a cell in vivo.

11. The method of claim 4, wherein the hsp70 is a mycobacterial hsp70.

12. The method of claim 4, wherein the moiety of interest is a protein or peptide.

13. The method of claim 12, wherein the protein or peptide is glycosylated.

14. The method of claim 4, wherein the antigen presenting cell is a human antigen presenting cell.

15. The method of claim 4, wherein the moiety of interest is covalently linked to the hsp70 by a peptide bond.

16. The method of claim 4, wherein the moiety of interest is covalently linked to the hsp70 by chemical conjugation.

17. The method of claim 4, wherein the cell is a cell in vivo.


SEQUENCE LISTING (7 sequences)

SEQ ID NO: 1 - protein, 8 aa, Unknown
  an immunodominant ovalbumin octapeptide; amino acids 258-276 of ovalbumin
SIINFEKL

SEQ ID NO: 2 - protein, 8 aa, Unknown
  amino acids 324-332 of vesicular stomatitus virus nucleoprotein
RGYVYQGL

SEQ ID NO: 3 - DNA, 31 bp, Unknown
  upstream primer oKS 63
gcccgggatccatggctcgtgcggtcgggat

SEQ ID NO: 4 - DNA, 28 bp, Unknown
  downstream primer oKS 79
gcggaattctcatcagccgagccggggt

SEQ ID NO: 5 - DNA, 35 bp, Unknown
  upstream primer oKS 83
gcggatccatatggtccttcagccaagctccgtgg

SEQ ID NO: 6 - DNA, 27 bp, Unknown
  downstream primer oKS 82
gcaggatccctcttccataacattaga

SEQ ID NO: 7 - DNA, 29 bp, Unknown
  downstream primer oKS 80
gctgaattcttactcttccataacattag